(19) United States
(12) Patent Application Publication
(10) Pub. No.: US 2010/0004540 A1
(43) Pub. Date: Jan. 7, 2010

(54) DUAL PATH PROCESSING FOR OPTIMAL SPECKLE TRACKING

(75) Inventor: Karl E. Thiele, Andover, MA (US)

Correspondence Address: PHILIPS INTELLECTUAL PROPERTY & STANDARDS, P.O. BOX 3001, BRIARCLIFF MANOR, NY, 10510, US

(73) Assignee: KONINKLIJKE PHILIPS ELECTRONICS N.V., Eindhoven (NL)

(21) Appl. No.: 12/447,969

(22) PCT Filed: Nov. 2, 2007
(86) PCT No.: PCT/IB07/54466

§ 371 Date: Apr. 30, 2009

Related U.S. Application Data
(60) Provisional application No. 60/864,259, filed on Nov. 3, 2006.

(51) Int. Cl. A61B 8/00

(52) U.S. Cl. 600/447

(57) ABSTRACT

This invention relates generally to an improved system and method that combines enhancing and mitigating techniques for speckle tracking, for obtaining a series of images of the movement of a target, such as tissue, over time. The method comprises steps of transmitting sound waves into the human body and outputting echoes of these sound waves; receiving and beamforming the echoes to produce scan line data; processing scan line data to display anatomical information using a method which reduces speckle; processing scan line data using a method or procedure which does not reduce speckle, and during one scan sequence, simultaneously acquiring the two scan line data, that data processed reducing speckle and that data processed without reducing speckle.

This invention relates generally to ultrasound imaging, and more particularly to ultrasound imaging using both enhanced and mitigated ultrasound speckle patterns.

Over the past decade, significant improvements to ultrasound image quality have resulted from advanced compounding techniques including frequency compounding and spatial compounding (SonoCT) techniques. These techniques work by mitigating ultrasound speckle, which is an artificial noise pattern related to the constructive/destructive interference patterns obtained from Raleigh scattered echoes.

Speckle is caused by random constructive and destructive interference associated with numerous small anatomic targets contained in the resolution cell of the ultrasound beam. These targets, or Raleigh Scatters, are, by definition, much shorter than the wavelength of the interrogating sound wave. The transmitted sound beam tends to be wide-band, which refers to the concept that this beam contains sound waves with various wavelengths. As is known by those skilled in the art, different wavelengths have different constructive and destructive interference patterns, and therefore have different speckle patterns. Much like the way a prism separates white light into its constituent wavelengths (colors), quadrature bandpass filters separate the returning sound echo into two groupings, one having shorter wavelengths, and the other having longer wavelengths. The two groupings will therefore have different interference patterns, and hence different speckle patterns.

Recently, there has been a desire to track both the velocity and displacements of blood and tissue (in 1D, 2D, and 3D space). Since the speckle pattern obtained from ultrasound imaging tends to track tissue and tissue displacements for short distances, accurate measures of tissue velocity and displacement can be calculated by cross-correlating a speckle pattern obtained over space with a similar speckle pattern obtained over time. These techniques have been referred to in the industry as 2D Speckle Tracking, and 3D Speckle Tracking. While optimal black and white (BW) image quality is obtained by mitigating the ultrasound speckle of the returning echo, optimal speckle tracking (displacements and velocities) is obtained when the ultrasound speckle is enhanced.

This invention relates generally to an improved system and method that combines enhancing and mitigating techniques for speckle tracking, for obtaining a series of images of the movement of a target, such as tissue, over time.

The method comprises steps of transmitting sound waves into the human body and outputting echoes of these sound waves; receiving and beamforming the echoes to produce scan line data; processing scan line data to display anatomical information using a method which reduces speckle; processing scan line data using a method or procedure which does not reduce speckle, and during one scan sequence, simultaneously acquiring the two scan line data, that data processed reducing speckle and that data processed without reducing speckle.

The foregoing and other objects, aspects, features, and advantages of the invention will become more apparent from the following description and from the claims.

In the drawings, like reference characters generally refer to the same parts throughout the different views. Also, the drawings are not necessarily to scale, emphasis instead generally being placed upon illustrating the principles of the invention.

FIG. 1 is an illustrative speckle image of a portion of a patient (tissue) produced from a low frequency quadrature band-pass filter.

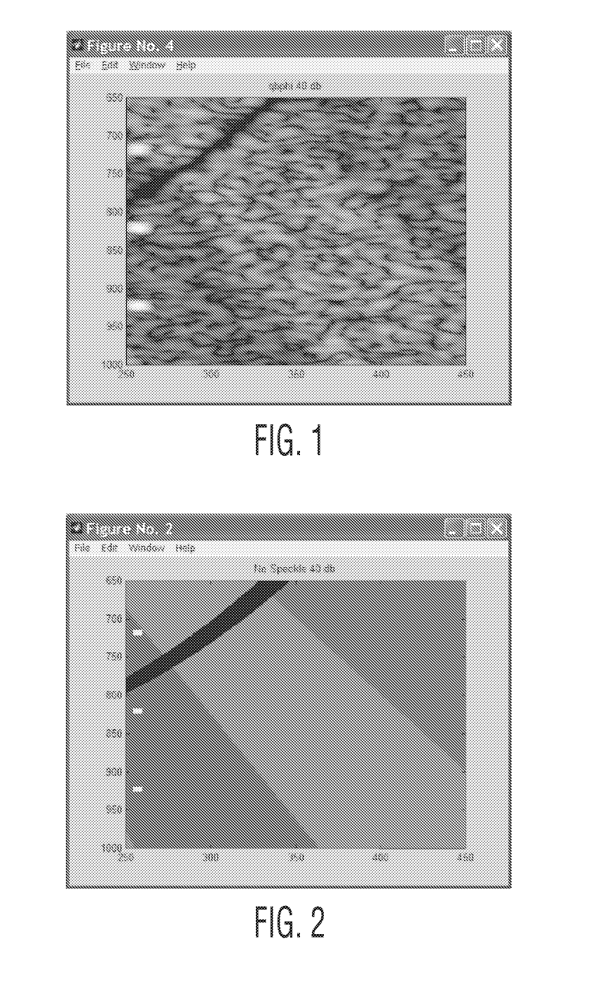

FIG. 2 is an illustrative synthetic phantom or truth image of a portion of a patient (tissue).

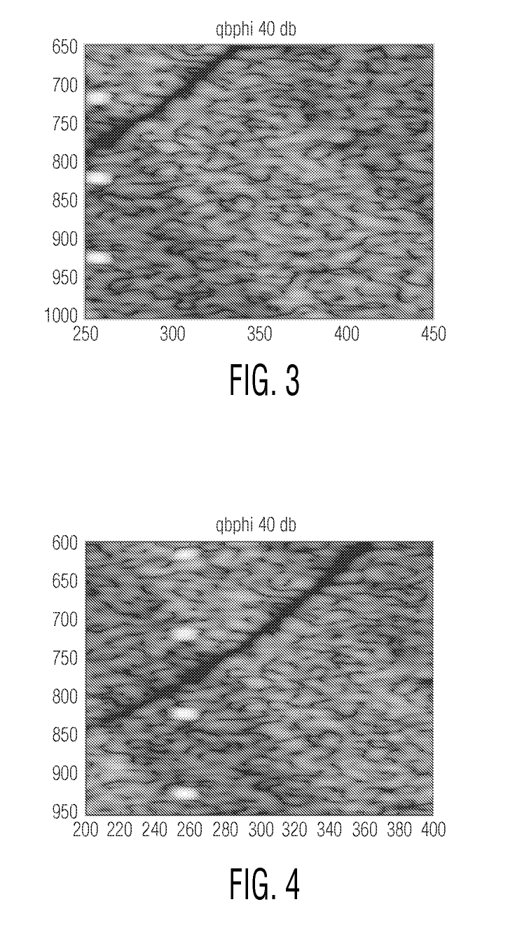

FIG. 3 is another illustrative speckle image of a portion of a patient (tissue) produced from a low frequency quadrature band-pass filter.

FIG. 4 is the tissue of FIG. 3 at a later time.

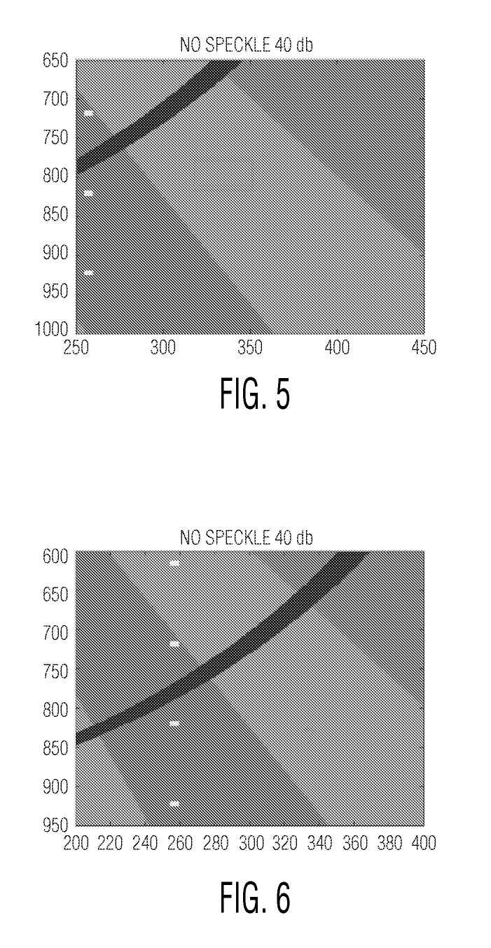

FIG. 5 is another illustrative synthetic phantom or truth image of a portion of a patient (tissue).

FIG. 6 is the truth image of the tissue shown in FIG. 5 at a later time.

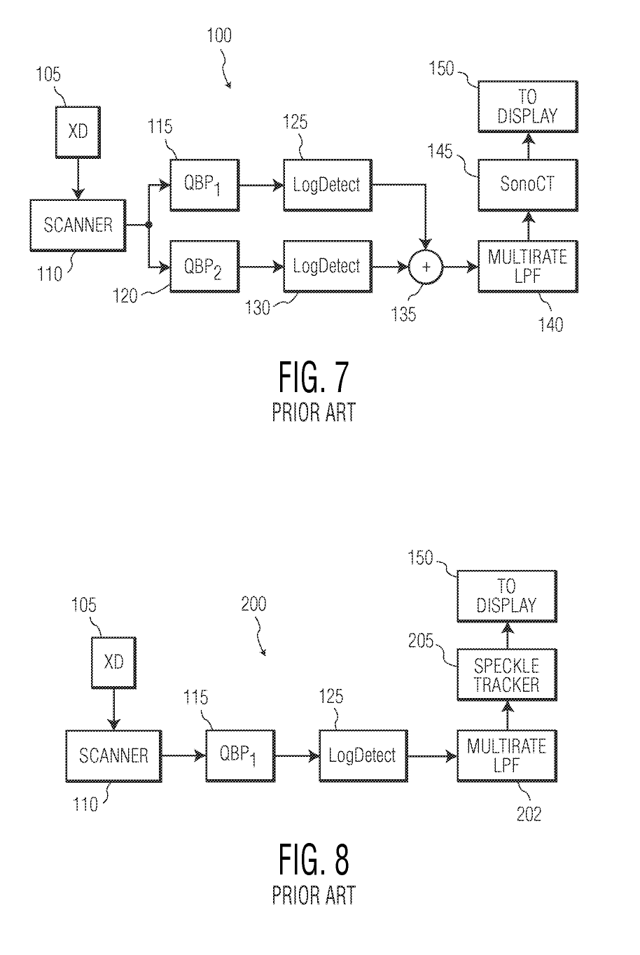

FIG. 7 is an illustrative schematic diagram of a prior art ultrasound imaging system configured to reduce speckle patterns.

FIG. 8 is an illustrative schematic diagram of a prior art ultrasound imaging system configured for optimal speckle tracking.

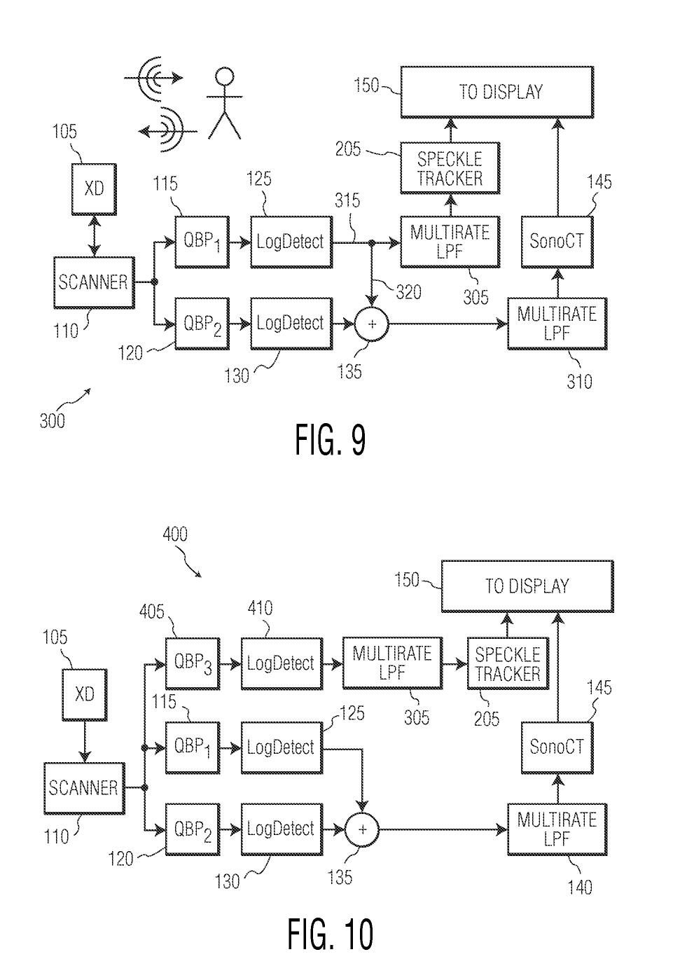

FIG. 9 is an illustrative schematic diagram of an ultrasound imaging system configured for optimal speckle tracking, according to one embodiment of the invention.

FIG. 10 is an illustrative schematic diagram of an ultrasound imaging system configured for optimal speckle tracking, according to another embodiment of the invention.

This invention relates to ultrasound imaging using both enhanced and mitigated ultrasound speckle patterns. The returning digitized echo corresponding to a single scan line is replicated and is sent to two separate processing paths. One path is optimized for black and white (BW) image quality, that is, reduced speckle. The other path is optimized for speckle tracking, that is, enhanced speckle.

When a target (e.g., human tissue) is illuminated with ultrasound waves, the target can constructively or destructively interfere with the ultrasound signal. An image of the target tissue appears grainy, or appears to have a texture. This grainy appearance is referred to as speckle. The speckle has nothing to do with the underlying data in the image. Speckle is simply arbitrary bumps or noise in the data that change as tissue moves. Thus, tracking speckle, that is, capturing speckle data over time, enables tracking of tissue movement and/or displacement or blood flow over time.

For example, speckle can be used to track the heart beats or movement through a cardiac cycle as follows. When blood flows properly through tissue, the tissue is soft. When blood does not properly flow through tissue, the tissue gets hard. The heart is sponge-like and has contractile properties. As the heart beats, it compresses and returns. However, dead or damaged tissue does not compress or move. Therefore, tracking the speckle patterns from ultrasound imaging of the heart over time enables one to track the beating or movement of the heart, or lack thereof.

FIG. 1 shows a speckle image of a portion of a patient, i.e. tissue, produced from a low frequency quadrature band-pass filter. FIG. 2 shows the synthetic phantom or truth image of a portion of a patient, i.e. tissue. In FIG. 2, all of the artificial speckle has been removed, making it easier to see the point targets on the left, the small black vessel in the upper left, and the subtle variations in the background gray levels (lower right). So FIG. 2 might be considered optimal from a 2D and/or anatomical presentation. However, if the tissue moved, and one wanted to detect this displacement relative change in position, FIG. 2 is not useful because it lacks any significant “texture” (especially in the lower right). Thus, detecting motion would be very difficult using FIG. 2.

In FIG. 3, an arbitrary region of tissue, illustrated by the grey box in the center, is identified for tracking. The grey box illustrates an area known as the Region Of Interest (ROI). FIG. 4 shows the same tissue, along with the ROI, but at a later time. As the ROI illustrates, the tissue has moved from its original location shown in FIG. 3. But more importantly, the texture, also known as the speckle or grain, is the same in both FIGS. 3 and 4. It is this texture that allows the various “speckle tracking” methods to determine how far any given tissue has moved.

FIG. 5 illustrates the same tissue, at the same time, as FIG. 3. However, in this case, all speckle has been eliminated. Again, a specific region of tissue (ROI, grey box) has been identified for tracking. Using the same speckle reduction techniques as was used in FIG. 5, FIG. 6 illustrates the same tissue at a later time. However, in FIG. 6, because all speckle has been removed, there is no way for any speckle tracking method to determine how far the desired tissue in the ROI of FIG. 5 has moved. Thus eliminating all speckle precludes tracking of the movement of tissue.

Referring to FIG. 7, a schematic diagram of a prior art ultrasound imaging system 100 configured to reduce speckle patterns is shown. The imaging system 100 includes an ultrasound transducer (XD) 105, a scanner 110, a first quadrature band-pass filter (QBP1) 115, a second quadrature band-pass filter (QBP2) 120, a LogDetect 125, a LogDetect 130, an averaging means 135, a multirate low-pass filter (LPF) 140, a SonoCT 145, and a display 150. In a preferred embodiment, it is expected that the scanner 110 has digitized the returning echoes, such that the subsequent processing steps are processed using digital hardware or using software as part of a CPU. The averaging means 135 can be as simple as summing the two outputs of the LogDetect 125 and 130, and halving the result.

In operation, the ultrasound transducer (XD) 105 is an ultrasound piezoelectric transducer that converts electrical signals to sound waves and back. The XD 105 scans a subject (patient) and produces ultrasound waves and outputs them to the scanner 110, which is a phase to wave beamformer that is used to direct and focus the ultrasound beam. The output of the scanner 110 is input to QBP1 115 and QBP2 120. The QBP1 115 and QBP2 120 are band-pass filters that each include a Hilbert transformer (1-3 MHz). The QBP1 115 is centered at 2 MHz, and the QBP2 120 is centered at 3 MHz. The QBP1 115 and QBP2 120 each output a complex analytic signal, referred to as an IQ signal, having a real Inphase signal, and a complex Quadrature signal. By taking the square root of the sum of the squares, one can calculate the envelope of the echo as: Envelope=\sqrt{I²+Q²}. The LogDetect 125 and LogDetect 130 receive the complex signal from the QBP1 115 and QBP2 120, respectively, and detect the envelope of the received complex signal, and then take the logarithm of the detected result. Note that the method of combining detected signals from different frequency bandpass filters is referred to as “frequency compounding”, and is a well-established technique in the ultrasound industry.

The averaging means 135 receives the logged-envelopes from the LogDetect 125 and LogDetect 130. The logged-envelopes from the LogDetect 125 and LogDetect 130 were derived from two different frequencies (e.g., 2 and 3 MHz respectively). Speckle changes as a function of frequency while the underlying signal remains the same. When the logged-envelopes are averaged together, the speckle is averaged out. The averaged signal is then input to the multirate low pass filter 140 and output to the SonoCT 145. Since the speckle can vary faster than the underlying mean signal, low pass filtering this data will further reduce the speckle variations. The multirate lowpass filter 140 also reduces the high spatial frequency information, thereby allowing the signal to be decimated. This reduces the numbers of samples per scan line from several thousand to only a few hundred. Having fewer samples decreases the computational burden of the downstream processing operations.

The SonoCt 145 is a compound imaging device, which obtains images from different viewing angles and then combines them into a single image. The speckle pattern varies with viewing angle. The purpose of inputting the output of the averaging means 135 into the multirate low-pass filter 140 and the SonoCt 145 is to further remove speckle from the ultrasound image. The output of the SonoCt 145 is then input to the display 150, such as a monitor.

Referring to FIG. 8, a schematic diagram of a prior art ultrasound imaging system 200 configured for optimal speckle tracking is shown. The imaging system 200 includes an ultrasound transducer (XD) 105, a scanner 110, a quadrature band-pass filter (QBP1) 115, a LogDetect 125, a multirate low-pass filter 202, a speckle tracker 205, and a display 150.

In operation, the XD 105 produces the ultrasound waves and outputs them to the scanner 110. The output of the scanner 110 is input to QBP1 115. The QBP1 115 outputs an IQ signal as described above. The LogDetect 125 receives the complex signal from the QBP1 115 and detects the envelope of the received complex signal. The envelope is then input to the multirate low-pass filter 202 and output to the speckle tracker 205. Unlike the multirate low-pass filter 140 used in FIG. 7 for optimal speckle reduction, this multirate low-pass filter 202 provides less smoothing and potentially less decimation. For optimal speckle tracking, it is desired that the speckle be enhanced, so that the prior techniques used to mask the speckle are now detrimental. The speckle tracker 205 is a cross-correlation device that tracks speckle at different points in time, that is, records image data as the target (e.g. tissue) moves to obtain the variation in the speckle. By cross-correlating the speckle at different points in time, the speckle tracker can calculate tissue displacements, tissue motions, and tissue compression. The output of the speckle tracker 205 is then input to the display 150.

There are numerous published methods of “speckle tracking” (e.g. U.S. Pat. No. 5,876,342, Chen, et al.). The method typically used for both tracking performance and speed is the “Normalized Cross-Correlation” method. It is defined as follows:

$$NCC ( dx , dy ) = \frac{{\sum }_{x ∈ ROI} {\sum }_{y ∈ ROI} {u}_{1} ( x , y ) {u}_{2} ( x - dx , y - dy )}{\sqrt{{\sum }_{x ∈ ROI} {\sum }_{y ∈ ROI} {u}_{1}^{2} ( x , y )} \sqrt{{\sum }_{x ∈ ROI} {\sum }_{y ∈ ROI} {u}_{2}^{2} ( x - , dx , y - dy )}}$$

where:
NCC is Normalized Cross Correlation function
dx,dy are Search space to determine how far the speckle has moved
xε=ROI: Summation is taken over x & y in the Region of Interest (ROI)
u₁ is Image at time 1
u₂ is Image at time 2

This equation is applied as follows:
1. First, the Region of Interest (ROI) that is selected for tracking in the first image is identified. Note that multiple ROIs can be selected, and that every pixel (or every voxel in a 3D volume) can be selected for tracking. This defines the ROI and the range of x and y in the first image: u₁.
2. Next, dx and dy are varied to displace the same sized ROI in the image observed at a later time: u₂.
3. For each dx and dy, the Normalized Cross Correlation (NCC) function is evaluated.
4. Steps 2 and 3 are repeated until a peak maximum value of the NCC is observed. An NCC value of 1.0 indicates the maximum correlation. The value of dx and dy at this peak value indicates how far the desired tissue, in the ROI, has moved.

As would be obvious to one skilled in the art, the lack of any texture or speckle variations within source ROI (in u1) or in the displaced ROI (in u2) would cause the NCC search algorithm to fail. A correlation value of 1.0 would be observed for all displaced values of dx and dy, and hence a peak could not be identified.

The present invention provides an improved system and method for combining data obtained from an image enhancing ultrasound signal path and data obtained from a speckle enhancing ultrasound signal path to obtain a series of images of the movement of tissue over time.

Referring to FIG. 9, a schematic diagram of a preferred embodiment of an ultrasound imaging system 300 configured for optimal speckle tracking is shown. The imaging system 300 includes an ultrasound transducer (XD) 105, a scanner/beamformer 110, a first quadrature band-pass filter (QBP1) 115, a second quadrature band-pass filter (QBP2) 120, a LogDetect 125, a LogDetect 130, an averaging means 135, a first multirate low-pass filter 305, a second multirate low-pass filter 310, a speckle tracker 205, a SonoCT 145, and a display 150.

In operation, the scanner 110 sends an electrical signal to the ultrasound transducer XD 105, which converts this electrical signal into sound waves. These sound waves are propagated into the body, and reflect off of various anatomic structures. The returning sound wave echoes are converted back into electric signals by the same ultrasound transducer XD 105, and then sent back to the scanner 110. The scanner 110 then processes these signals to isolate echoes from specific scan directions and depths, thereby ascertaining the anatomical structures at those locations.

The output of the scanner 110 is input to QBP1 115 and QBP2 120. In one embodiment, the QBP1 115 is centered at 2 MHz, and the QBP2 120 is centered at 3 MHz. The QBP1 115 and QBP2 120 each output an IQ signal, which is a complex signal from which signal noise is removed. The LogDetect 125 and LogDetect 130 receive the complex signal from the QBP1 115 and QBP2 120, respectively, and detect the envelope of the received complex signal. The averaging means 135 receives the signal envelopes from the LogDetect 125 via signal path 320 and LogDetect 130 and averages out the noise (speckle) from the images, as described above.

The averaged signal is then input to the multirate low-pass filter 310. The output of the multirate low-pass filter 310 is input to the SonoCT 145, which obtains images from different viewing angles and then combines them into a single image. The output of the SonoCt 145 is then input to the display 150.

The signal envelope from the LogDetect 125 is also input to the multirate low-pass filter 305 via signal path 315. The output of the multirate low-pass filter 305 is input to the speckle tracker 205, which tracks speckle at different points in time. As stated above, by cross-correlating the speckle at different points in time, the speckle tracker can calculate tissue displacements, tissue motions, and tissue compression. The output of the speckle tracker 205 is then input to the display 150.

The speckle data from the speckle tracker 205 and the image data from the SonoCT 145 are obtained by the display 150 simultaneously. This speckle data or “functional information” may be displayed side-by-side with the anatomical image data, either as graphs or as secondary images. In a preferred embodiment, this functional information can be overlayed or superimposed on top of the anatomical image data, for example using colors different from the anatomical image. Such images are often referred to in the ultrasound industry as “parametric images”.

Thus, the speckle data can superimposed on the image data to create parametric images, which allow the movement of the imaged tissue to be observed. The speckle data can be displayed in various colors based on its values. For example, in one embodiment, the “varying” speckle data, which indicates moving tissue, is displayed as green and the “non-varying” speckle data, which indicates non-moving tissue, is displayed as gray. Advantageously, when the “colored” speckle data is superimposed on the simultaneously obtained image data, the tissue that moves and the tissue that does not move can be observed. In addition to tissue movement, the obtained speckle data and image data can be used to observe blood flow. As blood flows, tissue expands and contracts over time, thus causing varying speckle data. If there is no blood flow, the obtained speckle data will not vary.

The direct output of the speckle tracker 205 provides motion and displacement information for the interrogated anatomy. This information can be used to determine numerous functional attributes. In one example, the displacement field can be differentiated with respect to time, to determine the velocity of different structures. In another example, spatial differences in the displacements can be used to calculate local strain. Such measures of strain can be exploited to differentiate between those portions of the heart muscle that are healthy and contracting, and those that are ischemic, dead, and non-contracting. In yet another example, the motion field can be used for timing analysis, to determine when different portions of the heart are contracting. In a normal healthy heart, all portions of the left ventricle tend to contract simultaneously. However, in a diseased heart with dissynchronous contraction, different portions of the myocardium contract at different times, leading to less efficient pumping.

All of the above-derived measures can be calculated either using dedicated hardware, or software running in a computer. Also, it might be possible to derive such measures either real-time (while the sound waves are being acquired), or non-real-time (post acquisition).

The above described inventive system and method is useful for detecting tumors in breast tissue. Current methods, such as mammography, are effective only when a tumor is surrounded by less dense tissue, such as in forty to fifty year old women. The present invention effectively detects tumors, that is, areas with no blood flow or tissue movement, regardless of surrounding tissue density, and can thus detect tumors in twenty to forty year old women.

The inventive method described above is also effective for finding infracted areas of the heart. Such areas have been damaged and have reduced blood flow, and therefore reduced movement, which can be tracked and observed.

Further, the present invention is quicker, safer, and less invasive than current diagnostic methods that involve ionizing radiation or the introduction of radioactive dyes.

A key limitation of the embodiment shown in FIG. 9 is that one of the QBP-filter-LogDetect processing banks (e.g., QBP filter 115 and LogDetect 125) is shared by both the reduced speckle image quality path and the optimal speckle-tracking path. Whereas this sharing may result in a lower cost implementation by requiring only two QBP-filter-LogDetect banks, it potentially compromises the performance of both the reduced speckle image quality path and the optimal speckle-tracking path. For example, it may be desirable for one of the paths to be configured for fundamental frequency operation (QBP filters have a center frequency close to the transmit frequency), and the other path configured for Tissue Harmonic Imaging (QBP filters have a center frequency twice that of the transmit frequency).

Referring to FIG. 10, in an alternative embodiment to address the performance restrictions of FIG. 9, a schematic diagram of an ultrasound imaging system 400 configured for optimal speckle tracking is shown. The imaging system 400 includes an ultrasound transducer (XD) 105, a scanner/beamformer 110, a first quadrature band-pass filter (QBP1) 115, a second quadrature band-pass filter (QBP2) 120, a third quadrature band-pass filter (QBP3) 405, a LogDetect 125, a LogDetect 130, a LogicDetect 410, an averaging means 135, a first multirate low-pass filter 305, a second multirate low-pass filter 310, a speckle tracker 205, a SonoCT 145, and a display 150.

In operation, the XD 105 converts the ultrasound waves into electrical signals and outputs them to the scanner 110. The output of the scanner 110 is input to QBP1 115, QBP2 120 and QBP3 405. In one embodiment, the QBP1 115 is centered at 2 MHz, and the QBP2 120 is centered at 3 MHz. This might relate to the scenario where the transmit frequency is centered at 2.5 MHz, and QBP1 115 and QBP2 120 are attempting to perform frequency compounding at the fundamental frequency which is close to the transmit frequency. These frequencies may have been chosen for optimal image quality and for optimal speckle reduction. In this same scenario, it may be concluded that optimal speckle tracking should be performed using Tissue Harmonic Imaging (see U.S. Pat. No. 5,879,303). In this case, it would be appropriate to have QBP3 405 centered at 5 MHz, which would be twice the frequency of the transmitted sound wave. The QBP1 115, QBP2 120, and QBP3 405 each output an IQ signal, which is a complex signal from which signal noise is removed. The LogDetect 125 and LogDetect 130 receive the complex signal from the QBP1 115 and QBP2 120, respectively, and detect the envelope of the received complex signal. The LogDetect 410 receives the complex signal from QBP3 405 and detects the envelope of the received complex signal.

The averaging means 135 receives the signal envelopes from the LogDetect 125 and LogDetect 130 and averages out the speckle from the images. The averaged signal is then input to the multirate low-pass filter 310. The output of the multirate low-pass filter 310 is input to the SonoCT 145. The output of the SonoCt 145 is then input to the display 150, such as a monitor.

At the same time, the signal envelope from the LogDetect 410 is input to the multirate low-pass filter 305. The output of the miltrate low-pass filter 305 is input to the speckle tracker 205. The output of the speckle tracker 205 is then input to the display 150.

All of the embodiments and block diagrams describe different methods of processing the same scan line, such that one path is optimized for optimal image quality and reduced speckle, and the second path is optimized for speckle tracking and enhanced speckle. A scan line is defined as a singular beam of sound interrogating a specific line of sight in the body, having dimensions of axial depth (e.g. in units of mm). Depending upon how this scan line is sequenced, different imaging modes and displays can be obtained. In one embodiment, the scan line can interrogate the same line of sight (referred to as M-Mode). In a second embodiment, the scan line can sequence through a tomographic slice in the body, referred to as 2D or B-Mode operation. In yet another embodiment, the scan line can vary both in the azimuth (lateral) and elevation dimensions, thereby scanning a volume (referred to as 3D or 4D imaging).

Also, as would be obvious to one of average skill in the art, this invention is applicable to any type of ultrasound transducer, including but not limited to single-element mechanical transducers, phased arrays, linears, curved-linear arrays (CLA's), 2D Matrix arrays, and Phased-array wobblers.

In yet another embodiment of this invention, it is assumed that the parallel processing paths are time-multiplexed, such that a single processing path is varied on a line-by-line basis, such that during one receive scan event, the path is optimized for speckle-tracking, and that for another receive scan event, which may be the same line of sight, the path is optimized for optimal image quality having mitigated speckle.

In yet another embodiment of this invention, the processing used for speckle-tracking involves using an RF filter for the bandpass filter and not having a LogDetect. Another embodiment of this invention involves reducing speckle by limiting a post detected low pass filter at a frequency cutoff below the frequency cutoff used n a speckle tracking path.

Variations, modifications, and other implementations of what is described herein may occur to those of ordinary skill in the art without departing from the spirit and scope of the invention. Accordingly, the invention is not to be defined only by the preceding illustrative description.

What is claimed is:

1. An ultrasound system comprising:
means for transmitting sound waves into a human body and outputting echoes of said sound waves;
means for receiving and beamforming the echoes to produce at least one scan line data;
first means for processing one of the scan line data to display anatomical information, said first means for processing including reducing speckle;
second means for processing one of the scan line data, said second means for processing not including reducing speckle; and
means for acquiring the one of the scan line data processed using said first means and the one of the scan line data processed using said second means simultaneously during one scan sequence.

2. The system of claim 1, wherein said first processing means and said second processing means process the same scan line data.

3. The system of claim 1, wherein said first processing means and said second processing means process different scan line data from the one scan sequence.

4. The system of claim 1, wherein the first processing means includes an RF bandpass filter.

5. The system of claim 1, wherein the second processing means includes an RF bandpass filter.

6. The system of claim 1, wherein the first processing means includes a means for detecting an envelope of the echoes.

7. The system of claim 6, wherein the first processing means includes a means for taking a logarithm of the detected envelope.

8. The system of claim 1, wherein reducing speckle is done using frequency compounding.

9. The system of claim 8, further comprising two or more filter banks, each filter bank comprising a detector and a bandpass filter having a unique response for each filter bank.

10. The system of claim 1, wherein reducing speckle is done by spatial compounding.

11. The system of claim 1, wherein the scan sequence interrogates at least one of a single line of sight, a plane, and a volume.

12. The system of claim 1, wherein the means for transmitting sound waves is selected from the group consisting of a phased-array, a Linear, a Curved Linear Array, a mechanical wobbler, and a 3D wobbler.

13. The system of claim 1, wherein the first processing means and the second processing means comprise an RF bandpass filter.

14. The system of claim 1, wherein the first processing means can be accomplished using dedicated hardware, or using software operating in a CPU.

15. The system of claim 1, wherein the second processing means can be accomplished using dedicated hardware, or using software operating in a CPU.

16. The system of claim 1, wherein the one of the scan line data processed using said second processing means can be superimposed on the anatomical information, using parametric imaging display techniques.

17. The system of claim 1, wherein the one of the scan line data processed using said second processing means is cross-correlated with data acquired from a previous scan sequence.

18. The system of claim 17, wherein the cross-correlated data can be used for strain, strain-rate, elastography, wall thickening, and contraction timing.

19. The system of claim 1, wherein reducing speckle is done by limiting a post detected low pass filter at a frequency cutoff below the frequency cutoff used in a speckle tracking path.

20. The system of claim 1, where the one scan sequence can be repeated for determine spatial displacements of a tissue over time.

21. A method for performing speckle tracking, said method comprising the steps of:
transmitting sound waves into a human body and outputting echoes of said sound waves;
receiving and beamforming the echoes to produce at least one scan line data;
processing one of the scan line data to display anatomical information, said processing including reducing speckle;
additional processing one of the scan line data, said additional processing not including reducing speckle; and
acquiring the one of the scan line data processed to display anatomical information and the one of the scan line data processed using additional processing simultaneously during one scan sequence.

22. A computer readable medium having computer readable program code for operating on a computer for performing speckle tracking, comprising:
transmitting sound waves into a human body and outputting echoes of said sound waves;
receiving and beamforming the echoes to produce at least one scan line data;
processing one of the scan line data to display anatomical information, said processing including reducing speckle;
additional processing one of the scan line data, said additional processing not including reducing speckle; and
acquiring the one of the scan line data processed to display anatomical information and the one of the scan line data processed using additional processing simultaneously during one scan sequence.
